(19) United States
(12) Patent Application Publication
(10) Pub. No.: US 2010/0204672 A1
(43) Pub. Date: Aug. 12, 2010

(54) SYSTEM AND METHOD FOR TREATING ISCHEMIC STROKE

(75) Inventors: Arthur John Lockhart, San Ramon, CA (US); Arani Bose, New York, NY (US); David Barry, Livermore, CA (US)

Correspondence Address: Penumbra Inc. c/o MoFo, 425 Market Street, San Francisco, CA, 94105-2482, US

(73) Assignee: Penumra, Inc., San Leandro, CA (US)

(21) Appl. No.: 12/370,288

(22) Filed: Feb. 12, 2009

(51) Int. Cl. A61M 1/00

(52) U.S. Cl. 604/500; 604/523; 604/151

(57) ABSTRACT

A thromboembolic removal system for treating ischemic stroke, including a guide and occlusion catheter, an aspiration catheter, an aspiration pump, and a thromboembolic separator. During aspiration of thromboembolic material through the aspiration catheter, the separator element is advanced from and retracted into the catheter lumen to break up or prevent clogs or flow restrictions formed by the thromboembolic material. Longitudinal channels in the separator allow aspiration through the aspiration catheter to continue even when the separator is disposed in the aspiration catheter lumen.

### BACKGROUND OF THE INVENTION

I. Field of the Invention

The present invention relates generally to the field of medical treatment and, more particularly, to a system and method for treating ischemic stroke which involves removing thromboembolic material from a cerebral artery of a patient.

II. Discussion of the Prior Art

Stroke is a leading cause of death and disability and a growing problem to global healthcare. In the US alone, over 700,000 people per year suffer a major stroke and, of these, over 150,000 people die. Even more disturbing, this already troubling situation is expected to worsen as the “baby boomer” population reaches advanced age, particularly given the number of people suffering from poor diet, obesity and/or other contributing factors leading to stroke. Of those who survive a stroke, approximately 90% will have long-term impairment of movement, sensation, memory or reasoning, ranging from mild to severe. The total cost to the US healthcare system is estimated to be over $50 billion per year.

Strokes may be caused by a rupture of a cerebral artery (“hemorrhagic stroke”) or a blockage in a cerebral artery due to a thromboembolism (“ischemic stroke”). A thromboembolism is a detached blood clot that travels through the bloodstream and lodges so as to obstruct or occlude a blood vessel. Between the two types of strokes, ischemic stroke comprises the larger problem, with over 600,000 people in the US suffering from ischemic stroke per year.

Ischemic stroke treatment may be accomplished via pharmacological elimination of the thromboembolism and/or mechanical elimination of the thromboembolism. Pharmacological elimination may be accomplished via the administration of thombolytics (e.g., streptokinase, urokinase, tissue plasminogen activator (TPA)) and/or anticoagulant drugs (e.g., heparin, warfarin) designed to dissolve and prevent further growth of the thromboembolism. Pharmacologic treatment is non-invasive and generally effective in dissolving the thromboembolism. Notwithstanding these generally favorable aspects, significant drawbacks exist with the use of pharmacologic treatment. One such drawback is the relatively long amount of time required for the thrombolytics and/or anticoagulants to take effect and restore blood flow. Given the time-critical nature of treating ischemic stroke, any added time is potentially devastating. Another significant drawback is the heightened potential of bleeding or hemorrhaging elsewhere in the body due to the thombolytics and/or anticoagulants.

Mechanical elimination of thromboembolic material for the treatment of ischemic stroke has been attempted using a variety of catheter-based transluminal interventional techniques. One such interventional technique involves deploying a coil into a thromboembolism (e.g. via corkscrew action) in an effort to ensnare or envelope the thromboembolism so it can be removed from the patient. Although an improvement over pharmacologic treatments for ischemic stroke, such coil-based retrieval systems have only enjoyed modest success (approximately 55%) in overcoming ischemic stroke due to thromboembolic material slipping past or becoming dislodged by the coil. In the latter case, the dislodgement of thromboembolic material may lead to an additional stroke in the same artery or a connecting artery.

Another interventional technique involves deploying a basket or net structure distally (or downstream) from the thromboembolism in an effort to ensnare or envelope the thromboembolism so it can be removed from the patient. Again, although overcoming the drawbacks of pharmacologic treatment, this nonetheless suffers a significant drawback in that the act of manipulating the basket or net structure distally from the occluded segment without angiographic roadmap visualization of the vasculature increases the danger of damaging the vessel. In addition, removing the basket or net structure may permit if not cause thromboembolic material to enter into connecting arteries. As noted above, this may lead to an additional stroke in the connecting artery.

A still further interventional technique for treating ischemic stroke involves advancing a suction catheter to the thromboembolism with the goal of removing it via aspiration (i.e. negative pressure). Although generally safe, removal via aspiration is only effective with relatively soft thrombus-emboli. To augment the effectiveness of aspiration techniques, a rotating blade has been employed to sever or fragment the thromboembolism, which may thereafter be removed via the suction catheter. While this rotating blade feature improves the effectiveness of such an aspiration technique, it nonetheless increases the danger of damaging the vessel due to the rotating blade.

Commonly-owned U.S Publication No. US2006/0058836, System and Method for Treating Ischemic Stroke, describes a separator device that enhances the effectiveness of the aspiration catheter while avoiding the risks associated with the prior art rotating blades and similar devices. The separator device is deployed from the distal end of an aspiration catheter positioned in the vessel from which the embolic material is to be removed. The separator may be advanced and retracted out of and into the aspiration catheter multiple times while vacuum pressure is applied to the aspiration catheter. Use of the separator device in this manner can facilitate aspiration of the thromboembolic material into the catheter in one of a variety of ways. First, if the separator is moved into contact with the thromboembolism in the vessel, movement of the separator into contact with the thromboembolism can loosen, separate, or soften pieces of thromboembolic material, such that pieces of the thromboembolism can be aspirated into the catheter. Second, advancing and retracting the separator serves to remove any clogs or flow restrictions within the lumen of the aspiration catheter that might be caused by the passage of thromboembolic material through the lumen. Additionally, during retraction of the separator, its proximal surface may push or plunge loosened material towards and/or into the distal end of the catheter for subsequent aspiration out of the body.

As described in the prior application, it is desirable to manufacture the separator and aspiration to have very close tolerances between the outer surface of the separator and the inner wall of the lumen. Such tolerances help to optimize the effect of the separator in removing clogs or flow restrictions from the lumen. However, the close tolerances can cause the separator to drastically reduce or briefly cut-off aspiration of material towards and through the lumen as the separator is withdrawn into the lumen. The present application discloses a thromboembolic removal system employing a separator device that improves upon the previously-described separator device by allowing aspiration to continue even when the separator is seated in the lumen.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a partial sectional side view of one embodiment of a thromboembolic removal system, including a guide catheter, an aspiration catheter, an aspiration pump, and a thromboembolic separator;

FIG. 2A is a perspective view of a distal portion of the separator of FIG. 1;

FIG. 2B is a plan view of the separator of FIG. 2A;

FIG. 2C is a cross-section view taken along the plane designated 2C-2C in FIG. 2A;

FIG. 3 is a cross-section view similar to FIG. 2C, showing an alternative separator embodiment;

FIGS. 4-7 are a sequence of drawings schematically illustrating use of the system of FIG. 1 within the cerebral vasculature.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The thromboembolic removal system disclosed herein boasts a variety of inventive features and components that warrant patent protection, both individually and in combination.

### System Features

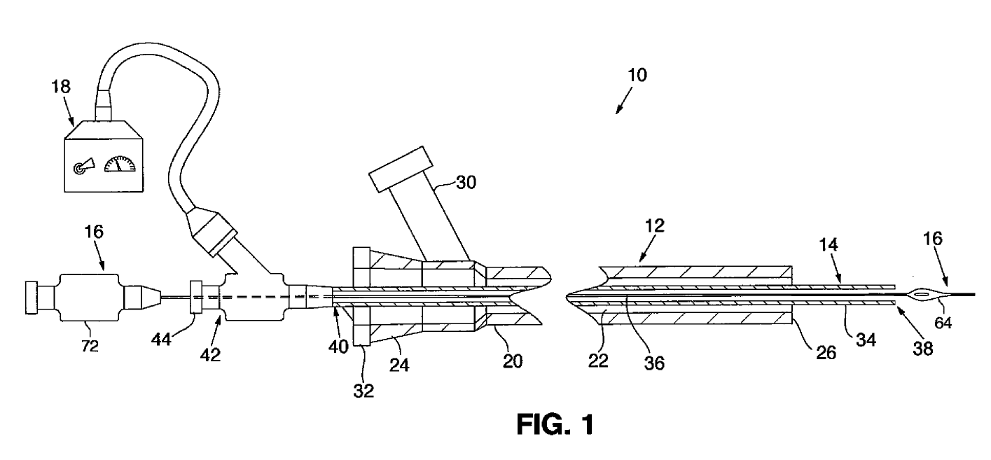

FIG. 1 illustrates an exemplary embodiment of a thromboembolic removal system 10. The thromboembolic removal system 10 includes an optional guide catheter 12, an aspiration catheter 14, a thromboembolic separator 16, and an aspiration pump 18. As will be described in greater detail below, the thromboembolic removal system 10 advantageously provides the ability to remove a thromboembolism from a cerebral artery within a patient while improving on features of the prior art. Further details can be found in commonly owned U.S. Publication No. US 2006/0058836, the disclosure of which is incorporated herein by reference.

The optional guide catheter 12 includes a tubular catheter member 20 having a main lumen 22 extending between a proximal end 24 and a distal end 26. The catheter member 20 may be constructed from any number of compositions having suitable biocompatibility and strength characteristics, and may be dimensioned in any number of suitable sizes and lengths depending upon the entry point into the vasculature, the location of the thromboembolism, variances in patient anatomy, and any extenuating circumstances. In an exemplary embodiment, the catheter member 20 may be constructed from nylon with embedded stainless steel braid and dimensioned having a length ranging from 70 cm to 120 cm and a diameter ranging from 5 French (0.065 inch) to 9 French (0.117 inch). A seal 32 is provided for passing the delivery and aspiration catheter 14 through the main lumen 22 of the guide catheter 12 in leak-free, hemostatic fashion. As another alternative, the catheter 14 can be introduced into the vasculature by a sheath.

The aspiration catheter 14 includes a tubular catheter element 34 having a main lumen 36 extending between a distal end 38 and a proximal end 40. The catheter member 34 may be constructed from any number of compositions having suitable biocompatibility and strength characteristics, and may be dimensioned in any number of suitable sizes and lengths depending upon the entry point into the vasculature, the location of the thromboembolism, variances in patient anatomy, and any extenuating circumstances. In an exemplary embodiment, the catheter member 34 may be constructed from pebax with embedded stainless steel braid and dimensioned having a length ranging from 130 cm to 170 cm and a diameter ranging from 2.5 French (0.032 inch) to 5 French (0.065 inch).

The aspiration catheter 14 also includes a hub assembly 42 coupled to the proximal end 40 for the purpose of coupling the lumen 36 to the aspiration pump 18. The hub assembly 42 also includes a seal 44 for allowing the passage of the thromboembolic separator 16 through the lumen 36 in leak-free, hemostatic fashion. The lumen is preferably coated with PTFE or another of the various suitable lubricious materials known in the art. A separator element 64 is located near the end of the separator 16.

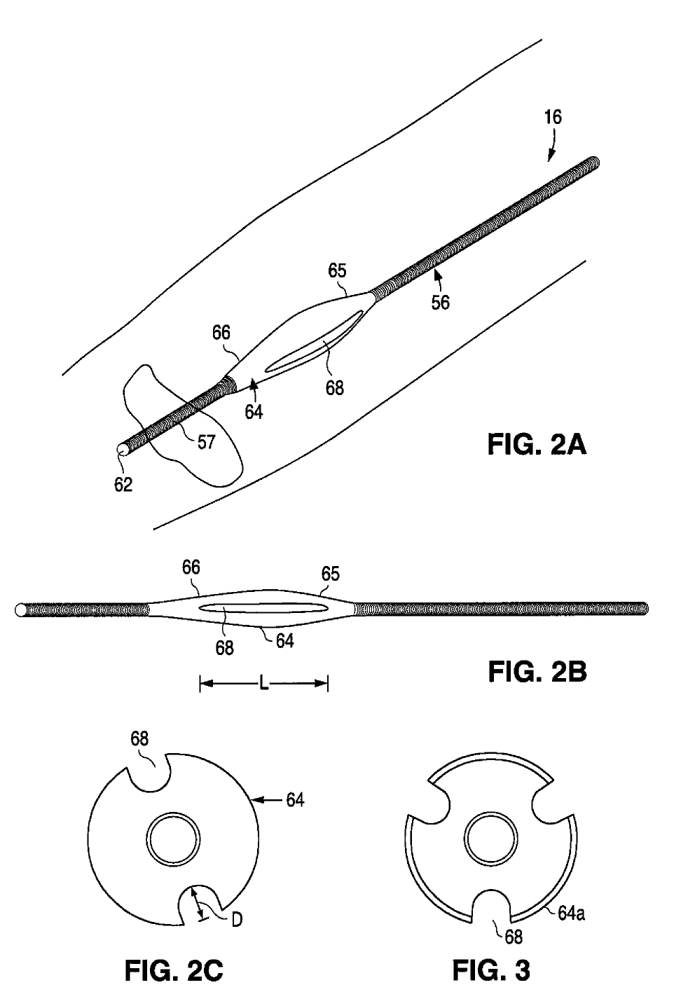

A first embodiment of a thromboembolic separator is shown in FIGS. 2A-2C. The thromboembolic separator 16 of the first embodiment includes an elongated element 56 having a proximal end (not seen in FIG. 2) and a distal end 57. The elongated element 56 may be constructed from any number of compositions having suitable biocompatibility and strength characteristics, and may be dimensioned in any number of suitable sizes and lengths depending upon the entry point into the vasculature, the location of the thromboembolism, variances in patient anatomy, and any extenuating circumstances. In an exemplary embodiment, the elongated element 56 may be constructed from stainless steel and/or Nitinol and dimensioned having a length ranging from 150 cm to 200 cm and a diameter ranging from 0.010 inch to 0.021 inch. A lubricious surface (e.g. a PTFE coating, hydrophilic coating, or other suitable coatings) may be applied to all or a portion of the elongate element 56 to facilitate movement of the element within the lumen of the delivery/aspiration catheter 14 and/or within the vasculature.

If desired, the elongate element 56 may be coiled along its length as shown in FIGS. 2A and 2B. Alternatively, the portion of the elongate element proximal to the separator element may be non-coiled with the distal section 57 portion of the elongate element, distal to the separator element, having a coiled configuration. In either case, the coiled distal section 57 has sufficient flexibility to prevent trauma to vascular tissues during advancement of the separator. The coil is preferably positioned around an inner mandrel or core (not shown) of a type commonly found in coiled guidewires.

The distal end of the elongated element 56 includes a generally blunt tip element 62 attached or forming part of the distal end thereof. The blunt nature of the tip element 60 is advantageously atraumatic such that it will not cause damage to the interior of the vasculature in the event it contacts a vessel wall during use.

Separator element 64 is formed of a polymeric material such as polyurethane or Pebax® polyether block amides, to name a few. The separator element 64 is preferably a solid, member having a first tapered portion 65 facing in the proximal direction, and a second tapered portion 66 oriented in a distal direction. The tapered portions 65, 66 may be contoured in a variety of ways. For example, portion 65 may have the conical configuration shown in FIGS. 2A and 2B, or it might be substantially planar or slightly convex as used for embodiments shown in Commonly-owned U.S Publication No. US2006/0058836, cited above.

The separator element 64 assists in removing any clogs or flow restrictions that may develop within the lumen of the aspiration catheter 34 (FIG. 1) due to the passage of thromboembolic material therethrough during aspiration. To facilitate this procedure, the separator element 64 and the catheter 14 are preferably provided with fairly tight tolerances between the diameter of the catheter lumen 36 and the greatest diameter of the separator element 64. For example, in one exemplary embodiment, the outer diameter of separator element 64 and the diameter of lumen 36 may differ by approximately 0.003-0.008 inches. It should be noted that the separator is preferably a fixed-diameter (non-collapsible) element.

A plurality of longitudinally extending channels or troughs 68 are formed in the separator element. The channels 68 are preferably oval shaped channels as shown in FIG. 2B, and include rounded bottom surfaces as shown in the FIG. 2C cross section. The channels are defined by smooth or radiused edges to avoid cutting or damage to vascular tissue in the event of contact between the vessel lumen and the separator.

The depth D of the channels (see FIG. 2c) is preferably in the range of 25 to 80 percent of the wall thickness of the separator element 64. In the case where the opposed ends of the separator element are tapered, then the length L of the channels (see FIG. 2B) is preferably in the range of 50 to 80 percent of the length of the separator element.

In the FIG. 2A-2C embodiment, two such channels are shown positioned 180° apart. Alternate embodiments may have different numbers of channels, and/or channels arranged with alternate spacings. For example, the alternate separator 64a of FIG. 3 includes three channels 68 spaced 120° apart.

In the illustrated embodiment, the separator element 64 is positioned on the coiled distal section 57 of the elongate element 56. The pitch of a portion of the coiled section 57 may be decreased in certain regions of the coiled distal section 57. Opening the spacing in the coil in this manner can facilitate adhesion between the polymeric material of the separator element and the coil material during the molding process. The spacing between the separator element 64 and the distal end of the elongate element 56 is preferably long enough to allow the distal-most portion of the elongate element sufficient flexibility to move atraumatically through the vasculature, but short enough to prevent folding of the distal-most portion during advancement of the elongate element 56. In an exemplary embodiment, the distal end of separator element 64 may be positioned approximately 3-9 mm from the distal end of the coil. It should be noted that the mandrel or core (not shown) within the coiled section 57 of the elongate element 56 might have a tapered diameter selected to enhance the flexibility of the coiled section.

Referring again to FIG. 1, a handle member 72 may be provided at the proximal end of the separator to provide a purchase point for a user to advance and/or manipulate the separator 16. The handle member 72 may be coupled to the elongated element 56 in any suitable fashion, including but not limited to providing a generally rigid extension (not shown) disposed within the elongated element 56 for the purpose of coupling the two components together. This coupling may be augmented or strengthened through the use of any number of adhesives or fusing techniques.

It will be appreciated that the guide catheter 12, the aspiration catheter 14, and/or the thromboembolic separator 16 may be provided with any number of features to facilitate the visualization of these elements during introduction and usage, including but not limited to having the distal regions equipped with radiopaque markers or filler materials for improved radiographic imaging. The system 10 may additionally be provided with instructions for use setting forth the various methods of use described herein, or equivalents thereof.

### System Use

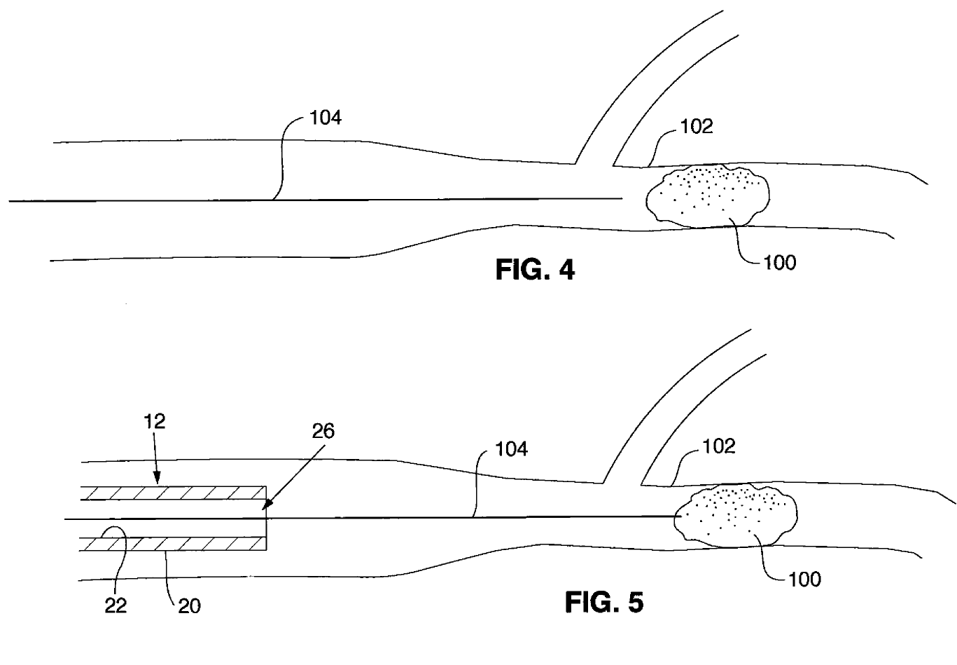

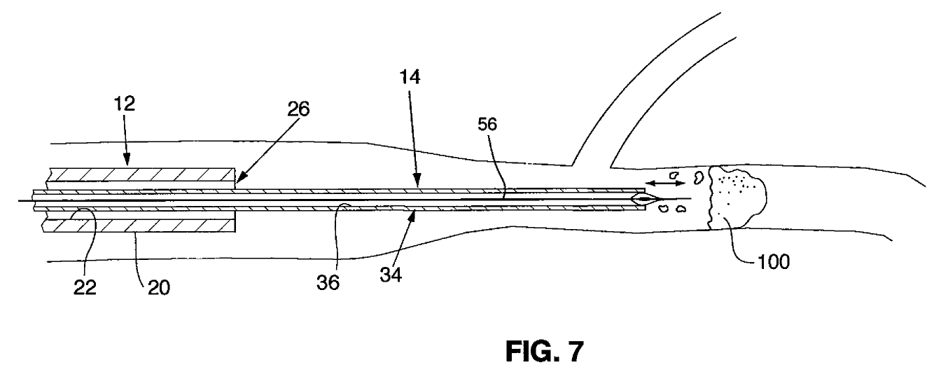

Methods of using the thromboembolic removal system 10 will now be described with reference to FIGS. 4-7. In a first exemplary method the thromboembolic removal system 10 is introduced into the patient's vasculature, such as via the Seldinger technique. FIG. 4 illustrates the first step of this process, which involves advancing a guide wire 104 to a point proximal to a thromboembolism 100. The guide wire 104 may comprise any number of commercially available guide wires, the operation of which is well known in the art. However the elongate member 56 of the separator 16 may be used instead of the guidewire 104.

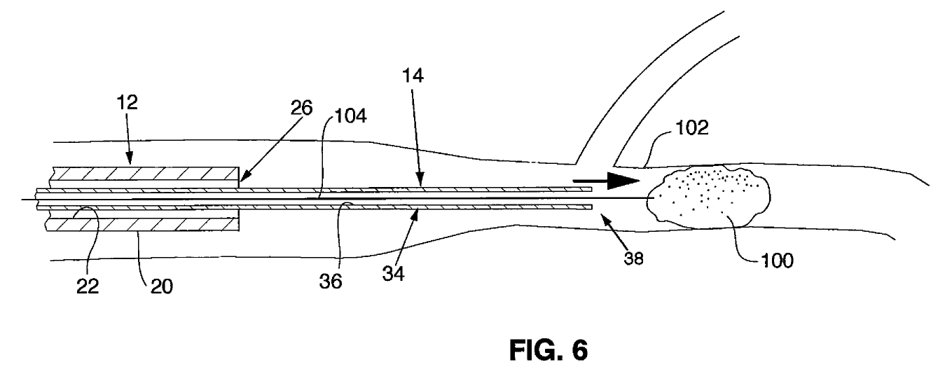

FIG. 5 illustrates a second step, which involves advancing the guide catheter 12 over the guide wire 104 (or the separator member 56) to a point proximal to the thromboembolism. As shown in FIG. 6, the aspiration catheter 14 is then advanced through the guide catheter 12 such that the distal end 38 of the aspiration catheter 14 is positioned at a point proximal to the thromboembolism 100. This is preferably facilitated by advancing the aspiration catheter 14 over the guide wire 104 (or the separator 16 when used in place of a guide wire). If the separator 16 was not used as the guide wire, the guide wire is next withdrawn and the separator 16 is introduced into the aspiration catheter 14.

At this point, the aspiration pump 18 (FIG. 1) may be activated to establish negative pressure within the aspiration catheter 14. In this fashion, negative pressure will be created within the cerebral artery 102 and exerted upon the thromboembolism 100, causing a reversal of blood flow in the vessel in the region surrounding the distal end of the aspiration catheter. The separator element 64, or a portion thereof, is advanced slightly from the lumen of the aspiration catheter, and is advanced and retracted several times within the distal end of the lumen 36 of the aspiration catheter 14.

Advancing and retracting the separator element 64 within the lumen 36 of the aspiration catheter serves to remove any clogs or flow restrictions that form within the lumen due to the passage of thromboembolic material through the lumen 36. When the separator element 64 is positioned within the lumen, the channels 68 in the separator 64 fluidly couple the lumen of the catheter to the blood vessel. This allows advancement and retraction of the separator into and out of the lumen 36 while preventing a nearly complete obstruction of the aspiration catheter. The embolic material can thus continue flowing towards and through the aspiration catheter in a continuous fashion.

In some procedures, the separator may be advanced into contact with a portion of the thromboembolism, or completely through the thromboembolism 100. This will serve to break up or otherwise soften the thromboembolism 100, or to bias the thromboembolic material towards the aspiration catheter. Selective advancement of the separator element 64 through the thromboembolism and retraction of the separator element into the aspiration catheter 14, preferably in combination with aspiration, can additionally be used to carry small “bites” of the thromboembolic material into the catheter 14. For example, the separator element 64 may be passed through the thromboembolic material, displacing some material and thus forming a channel in the material as it moves distally. Once the separator element is positioned further into, or distally of, the thromboembolism, some of the displaced material may flow back into this channel. Subsequent retraction of the separator element 64 through the material (e.g. through the re-filled channel) will then draw some of the material into the catheter 14.

An additional advantage to the channels is that they reduce the likelihood that any thrombus that had been previously drawn into the lumen will be pushed back out of the distal end of the lumen when the separator element is pushed out the distal end of the lumen.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternative falling within the spirit and scope of the invention.

Any and all applications referred to herein are hereby incorporated herein by reference.

We claim:

1. A method for removing thromboembolic material from a blood vessel in a patient, the method comprising the steps of:
providing a catheter having a lumen, the lumen including a distal opening with a fixed inner diameter, and further providing an elongate member extendable through the lumen and having a separator element thereon, the separator element comprising a solid geometric member having a plurality of longitudinal channels;
inserting the catheter into a blood vessel and positioning the catheter proximally of a body of thromboembolic material;
applying vacuum pressure through the lumen to draw thromboembolic material towards and into the lumen;
while applying the vacuum, reciprocating the separator element a plurality of times between a first position within the distal opening and a second position distal to the distal opening, wherein the longitudinal channels fluidly couple the lumen to the blood vessel when the separator element is in the first position.

2. The method of claim 1, wherein the reciprocating step prevents thromboembolic material entering the lumen from obstructing aspiration of additional material into the lumen.

3. The method of claim 1, wherein the reciprocating step plunges thromboembolic material into the lumen.

4. The method of claim 1, wherein the reciprocating step breaks up thromboembolic material accumulating in the lumen.

5. A system for removing thromboembolic material from a cerebral blood vessel, the system comprising:
an elongate catheter proportioned for insertion into a cerebral blood vessel, the catheter having a lumen extending therethrough, the lumen including a distal end having a fixed inner diameter;
an aspiration source fluidly coupled to the lumen; and
an elongate member extendable through the lumen and having a separator element thereon, the separator element comprising a solid geometric member having a plurality of longitudinal channels.

6. The system of claim 5, where the separator element is moveable between a first position within the lumen and a second position distal to the lumen, wherein the longitudinal channels provide a fluid path between the lumen and a region distal to the lumen when the separator element is in the first position.

7. The system of claim 5, wherein the elongate member is a coil member.

8. The system of claim 5, wherein the longitudinal channels extend parallel to the longitudinal axis of the elongate member.

9. The system of claim 5, wherein the separator includes only two longitudinal channels.

10. The system of claim 5, wherein the separator includes only three longitudinal channels.

11. A system for removing thromboembolic material from a cerebral blood vessel comprising;
a catheter having proximal and distal ends and a central lumen running the length thereof;
a vacuum pump fluidly coupled to the lumen at the proximal end of the catheter to apply suction thereto; and
a elongated member slidably received within the lumen, said member including a region having an increased diameter defining a separator element, said separator element being located near to but spaced from the distal end of the member, with the maximum dimension of the outer diameter of the separator element being close to the inner diameter of the lumen, and wherein said separator element includes at least one longitudinal trough extending along an axis parallel to the member, said trough cooperating with the inner wall of the lumen to define an aspiration channel when the member is positioned so that the separator element is within the catheter.

12. A system as recited in claim 11, wherein the distal and proximal ends of the separator element are tapered.

13. A system as recited in claim 11, wherein the separator element includes a pair of opposed troughs.

14. A system as recited in claim 11, wherein the separator element includes three troughs disposed equally about the circumference thereof.

15. A system as recited in claim 11, wherein the inner surface of the trough is rounded.

16. A system as recited in claim 11, wherein the difference between the maximum outer diameter of the trough and the inner diameter of the lumen is 0.003 to 0.008 inches.
